(19) United States
(12) Patent Application Publication
(10) Pub. No.: US 2025/0281239 A1
(43) Pub. Date: Sep. 11, 2025

(54) SYSTEMS AND METHODS FOR PROVIDING AUTOMATED TRAINING FOR MANUALLY CONDUCTED PROCEDURES

(71) Applicant: THE PENN STATE RESEARCH FOUNDATION, University Park, PA (US)

(72) Inventors: Scarlett Miller, State College, PA (US); Jason Moore, State College, PA (US); Hang-Ling Wu, State College, PA (US); Dailen Brown, State College, PA (US); Aayod Kaul, State College, PA (US)

(73) Assignee: THE PENN STATE RESEARCH FOUNDATION, University Park, PA (US)

(21) Appl. No.: 19/032,683

(22) Filed: Jan. 21, 2025

Related U.S. Application Data
(60) Provisional application No. 63/562,894, filed on Mar. 8, 2024.
(60) Provisional application No. 63/634,605, filed on Apr. 16, 2024.

(51) Int. Cl. A61B 34/10; G06F 3/01; G09B 23/28

(52) U.S. Cl. CPC A61B 34/10; G06F 3/016; G09B 23/285; G09B 23/286; A61B 2034/102; A61B 2034/105

(57) ABSTRACT

Systems and methods for providing automated training. An automated training system includes hardware for receiving images from an imaging device arranged such that a field of view of the imaging device includes a tray supporting tools and a training surface having a simulated subcutaneous area, determining a location, a position, and an identification of the tools supported on the tray, receiving an input from a position tracking system that corresponds to insertion characteristics of a tool into the training surface, determining, based on the insertion characteristics and the images of the training surface, a positioning and an orientation of at least a portion of the tool within the simulated subcutaneous area, and provide feedback regarding the positioning and orientation of the tool.

### CROSS REFERENCE TO RELATED APPLICATION

This application claims the benefit of priority to U.S. Provisional patent application Ser. No. 63/634,605, filed on Apr. 16, 2024 and entitled “A SYSTEM AND A METHOD FOR COMPUTER VISION DETECTION IN MEDICAL SIMULATION TRAINING,” and also claims the benefit of priority to U.S. Provisional Application No. 63/562,894, filed Mar. 8, 2024 and entitled “METHOD FOR MEASURING USER INTERACTION WITH MEDICAL TOOLS USING ARTIFICIAL INTELLIGENCE,” the entire contents of both are incorporated herein in their respective entireties.

### GOVERNMENT SUPPORT

This invention was made with government support under Grant No. HL127316 awarded by the National Institutes of Health. The Government has certain rights in the invention.

### BACKGROUND

### Field

The present disclosure generally relates to a training system, and in particular, to a dynamic haptic robotic training system.

### Technical Background

Various manually conducted procedures, such as procedures conducted by medical personnel on subjects, oftentimes are completed as a series of very distinct steps. However, the specific anatomy of a subject may necessitate deviations from the series of steps to ensure effectiveness of the procedure. For example, Central Venous Catheterization (CVC) is a medical procedure where medical personnel attempt to place a catheter in the jugular, subclavian, or femoral vein of a subject. While useful, this procedure can subject individuals undergoing the procedure to some adverse effects. Traditionally, training is performed on CVC manikins. These traditional CVC training systems range from low-cost homemade models to “realistic” manikins featuring an arterial pulse and self-sealing veins (e.g. Simulab CentralLineMan® controlled through a hand-pump). While these simulators allow multiple needle insertion and practice trials without consequence, they are static in nature and may not vary an anatomy of the subject to give the practitioner experience in a variety of potential real world scenarios.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes an automated training system. The automated training system also includes an imaging device arranged to capture one or more images of a tray supporting one or more tools and a training surface having a simulated subcutaneous area. The system also includes a dynamic haptic manual control device. The system also includes a position tracking system. The system also includes a display. The system also includes a computing device communicatively coupled to the imaging device, the dynamic haptic manual control device, the position tracking system, and the display. The computing device is configured to receive the one or more images from the imaging device, determine a location, a position, and an identification of the one or more tools supported on the tray, receive an input from the position tracking system, the input corresponding to insertion characteristics of: a tool of the one or more tools into the training surface, and/or the dynamic haptic manual control device. The computing device is also configured to determine, based on the insertion characteristics and the one or more images of the training surface, a positioning and an orientation of at least a portion of the tool and/or the dynamic haptic manual control device within the simulated subcutaneous area, and provide feedback, via the display and/or the dynamic haptic manual control device, regarding the positioning and orientation of the tool and/or the dynamic haptic manual control device. Other embodiments of this aspect include corresponding computer systems, apparatus, and computer programs recorded on one or more computer storage devices, each configured to perform the actions of the methods.

One general aspect includes an automated training system. The automated training system also includes a computing device. The system also includes a non-transitory, computer-readable storage medium communicatively coupled to the computing device, the non-transitory, computer-readable storage medium may include one or more programming instructions thereon that, when executed, cause the computing device to: receive one or more images from an imaging device arranged such that a field of view of the imaging device includes a tray supporting one or more tools and a training surface having a simulated subcutaneous area, determine a location, a position, and an identification of the one or more tools supported on the tray, receive an input from a position tracking system, the input corresponding to insertion characteristics of: a tool of the one or more tools into the training surface, and/or a dynamic haptic manual control device. The system also includes determine, based on the insertion characteristics and the one or more images of the training surface, a positioning and an orientation of at least a portion of the tool and/or the dynamic haptic manual control device within the simulated subcutaneous area, and provide feedback, via a display and/or the dynamic haptic manual control device, regarding the positioning and orientation of the tool and/or the dynamic haptic manual control device. Other embodiments of this aspect include corresponding computer systems, apparatus, and computer programs recorded on one or more computer storage devices, each configured to perform the actions of the methods.

One general aspect includes a method of providing an automated training system. The method also includes receiving, by a computing device, one or more images from an imaging device arranged such that a field of view of the imaging device includes a tray supporting one or more tools and a training surface having a simulated subcutaneous area. The method also includes determining, by the computing device, a location, a position, and an identification of the one or more tools supported on the tray. The method also includes receiving, by a computing device, an input from a position tracking system, the input corresponding to insertion characteristics of: a tool of the one or more tools into the training surface, and/or a dynamic haptic manual control device. The method also includes determining, based on the insertion characteristics and the one or more images of the training surface, a positioning and an orientation of at least a portion of the tool and/or the dynamic haptic manual control device within the simulated subcutaneous area. The method also includes providing feedback, via a display and/or the dynamic haptic manual control device, regarding the positioning and orientation of the tool and/or the dynamic haptic manual control device. Other embodiments of this aspect include corresponding computer systems, apparatus, and computer programs recorded on one or more computer storage devices, each configured to perform the actions of the methods.

One general aspect includes a dynamic haptic syringe apparatus for providing automated training. The dynamic haptic syringe apparatus also includes a hollow syringe body having an open proximal end and a distal end. The apparatus also includes a syringe plunger having a plunger body defining a distal end and a proximal end, the syringe plunger received within the open proximal end of the hollow syringe body and movable within the hollow syringe body. The apparatus also includes a hub removably coupled to the distal end of the hollow syringe body. The apparatus also includes a telescopic needle assembly coupled to the hub, the telescopic needle assembly comprising a hollow outer needle having an open distal end and an open proximal end having an end effector disposed thereon, and an inner needle comprising an elongate body having a distal end and a proximal end rigidly coupled to the hub, the elongate body received within the hollow outer needle, the inner needle fixed in position and the hollow outer needle movable between an extended position and a retracted position, wherein in the extended position, the proximal end of the hollow outer needle is disposed within the hub or adjacent to the hub and in the retracted position, the proximal end of the hollow outer needle extends through the hub into the hollow syringe body. The apparatus also includes one or more slip flexures disposed within the hollow syringe body, the one or more slip flexures engagable with the end effector on the proximal end of the hollow outer needle to provide simulated feedback.

These and additional features provided by the aspects described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like structure is indicated with like reference numerals and in which:

FIG. 1 depicts a block diagram of an illustrative automated training system according to one or more aspects of the present disclosure;

FIG. 2A depicts a perspective view of an illustrative automated training system that includes an imaging device, a computer vision imaging surface, a dynamic haptic manual control device, a position tracking system, and a user interface according to one or more aspects of the present disclosure;

FIG. 2B depicts various additional components of the computer vision imaging surface depicted in FIG. 2A according to one or more aspects of the present disclosure;

FIG. 2C schematically depicts a cross-sectional side view of a portion of the computer vision imaging surface of an automated training system with a dynamic haptic manual control device inserted therein according to one or more aspects of the present disclosure;

FIG. 3 depicts a top-down view of an illustrative computer vision imaging surface of an automated training system according to one or more aspects of the present disclosure;

FIG. 4A depicts a side view of an illustrative dynamic haptic manual control device according to one or more aspects of the present disclosure;

FIG. 4B illustrates an aspect of the subject matter in accordance with one embodiment.

FIG. 4C depicts the dynamic haptic manual control device of FIG. 4A when rotated 90 degrees around a longitudinal axis thereof;

FIG. 5 is a detailed perspective view of a selection ring of a dynamic haptic manual control device according to one or more aspects of the present disclosure;

FIG. 6 depicts engagement of an end effector of an inner needle of a dynamic haptic manual control device with a slip flexure according to one or more aspects of the present disclosure;

FIG. 7 schematically depicts engagement of an end effector of an inner needle with a slip flexure according to one or more aspects of the present disclosure;

FIG. 8 depicts side views of various shapes of a slip flexure used in a dynamic haptic manual control device according to one or more aspects of the present disclosure;

FIG. 9 graphically depicts (a) haptic profiles for various slip flexure geometries with a single layer of grade 70 silicone and (b) haptic profiles for a consistent geometry under various layer counts and material grades according to one or more aspects of the present disclosure;

FIG. 10 depicts a cutaway perspective view of an illustrative syringe plunger according to one or more aspects of the present disclosure;

FIG. 11 schematically depicts a user interface showing annotated image labels of components detected on the computer vision imaging surface of FIG. 3;

FIG. 12 schematically depicts another user interface showing annotated image labels of components detected on the computer vision imaging surface of FIG. 3;

FIG. 13A depicts a flow diagram of an illustrative method of providing automated training to a user using the automated training system according to one or more aspects of the present disclosure;

FIG. 13B depicts a flow diagram of illustrative steps for determining an identification of tools according to one or more aspects of the present disclosure;

FIG. 13C depicts a flow diagram of illustrative steps for determining a position and/or orientation of tools and/or a dynamic haptic manual control device according to one or more aspects of the present disclosure; and

FIG. 13D depicts a flow diagram of illustrative steps for providing feedback according to one or more aspects of the present disclosure.

### DETAILED DESCRIPTION

The present disclosure generally relates to systems and methods that provide automated training for a user by combining aspects of image recognition, device position tracking (e.g., via electromagnetic sensors or the like), use of tools that provide haptic feedback, and a user interface that simulates real-world conditions. The systems and methods described herein can be effective in providing a user with an ability to practice technique for a particular procedure under real-world conditions that can be varied, while at the same time not exposing the user to conditions that might have adverse effects. While the present disclosure discusses these systems and methods specifically with respect to a medical procedure such as a CVC procedure, the systems and methods are not limited to such. That is, the systems and methods described herein can be adapted to other medical procedures, non-medical procedures, and/or the like.

Aspects described herein can be used, for example, to measure a user's interaction with a medical tool or the like, such as an endoscope. Aspects described herein can allow a user to interact with the medical tool while artificial intelligence is utilized to measure and interpret the interaction. Specifically, aspects described herein allow for an imaging device to record images, video, and/or the like to measure endoscopic knob rotation angle in real time, which allows for more simple and effective medical training. Unlike existing systems that utilize user inputs using specialized equipment, aspects of the present disclosure can be used to gather information with an imaging device.

Aspects described herein further relate to a training system for medical tool identification using machine learning. The systems and methods described herein produce labels from Ultraviolet (UV) light to gather training data for machine learning to identify a location of the medical tools. This is in contrast to existing systems, which track a specific order in which a user uses tools and is unable to track tools that are used out of a predetermined order.

Aspects described herein further relate to the use of a haptic syringe that uses compliant mechanisms. The systems and methods descried herein allows a user to be exposed to diverse subject profiles through a dynamic syringe that can be characterized to mimic various subject profiles, such as, for example, skin thickness, adipose tissue depth, and/or the like.

Endoscopic procedures, such as, for example, colonoscopies, laparoscopies, mediastinoscopies, colposcopies, sigmoidoscopies, cystoscopies, thoracoscopies, bronchoscopies, laryngoscopies, arthroscopies, or the like, are typically completed by highly skilled practitioners that are able to successfully maneuver the endoscope. Manikins offer highly realistic training relative to existing robotic training systems, but lack automated learning feedback. To acquire automated feedback during manikin training, the systems and methods described herein are able to read a user's manipulation of the endoscope control handle position and/or various other tools by utilizing a trained machine learning algorithm that is able to accurately measure the position of the tools and/or portions thereof (e.g., a control handle or the like) from images that are collected via the systems as described herein. Similarly, the devices, systems, and methods described herein can be applied to measure various endoscope and/or other tool manipulation movements during a simulated procedure. It should be appreciated that while endoscopic procedures are discussed herein as one example, the present disclosure is not limited solely to endoscopic procedures. That is, the devices, systems, and methods described herein may also be utilized for training of various other procedures, including medical and non-medical procedures, particularly procedures where a user is taught or practices various techniques for that procedure. For example, the devices, systems, and methods described herein may be used for various medical procedures such as, but not limited to, tracheostomy/tracheotomy procedures, procedures involving tissue incisions, biopsy procedures, needle insertion procedures, and/or the like. In another example the devices, systems, and methods described herein may be used for non-medical procedures such as, but not limited to, manufacturing procedures, inspection procedures, repair procedures, research procedures, law enforcement procedures, and/or the like. Other uses of the devices, systems, and methods described herein may be apparent from the present disclosure.

As used herein, the term “manikin” generally refers to anatomical models that are specifically used for medical training or practice. In some embodiments, a manikin may replicate an entire mammal body. In other embodiments, a manikin may only replicate a portion of a mammal body. For example, in the context of the present disclosure, a manikin may be used to replicate one or more subcutaneous areas of a mammal, such as a human patient or non-human patient. However, the present disclosure is not limited solely to manikins that replicate subcutaneous areas.

As used herein, connection references (e.g., attached, coupled, connected, and joined) can include intermediate members between the elements referenced by the connection reference and/or relative movement between those elements unless otherwise indicated. As such, connection references do not necessarily infer that two elements are directly connected and/or in fixed relation to each other. As used herein, stating that any part is in “contact” with another part is defined to mean that there is no intermediate part between the two parts.

Unless specifically stated otherwise, descriptors such as “first,” “second,” “third,” etc., are used herein without imputing or otherwise indicating any meaning of priority, physical order, arrangement in a list, and/or ordering in any way, but are merely used as labels and/or arbitrary names to distinguish elements for ease of understanding the disclosed examples. In some examples, the descriptor “first” can be used to refer to an element in the detailed description, while the same element can be referred to in a claim with a different descriptor such as “second” or “third.” In such instances, it should be understood that such descriptors are used merely for identifying those elements distinctly that might, for example, otherwise share a same name.

As used herein, the phrase “communicatively coupled,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

When introducing elements of various embodiments of the present disclosure, the articles “a,” “an,” “the,” and “said” are intended to mean that there are one or more of the elements. The terms “comprising,” “including,” and “having” are intended to be inclusive and mean that there can be additional elements other than the listed elements.

As used herein, the terms “system,” “unit,” “module,” “device,” “component,” etc., can include a hardware and/or software system that operates to perform one or more functions. For example, a module, unit, or system can include a computer processor, controller, and/or other logic-based device that performs operations based on instructions stored on a tangible and non-transitory computer readable storage medium, such as a computer memory. Alternatively, a module, unit, or system can include a hard-wires device that performs operations based on hardwired logic of the device. Various modules, units, engines, and/or systems shown in the attached figures can represent the hardware that operates based on software or hardwired instructions, the software that directs hardware to perform the operations, or a combination thereof.

Approximating language, as used herein throughout the specification and claims, is applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value, or the precision of the methods or machines for constructing or manufacturing the components and/or systems. For example, the approximating language may refer to being within a ten percent (10%) margin.

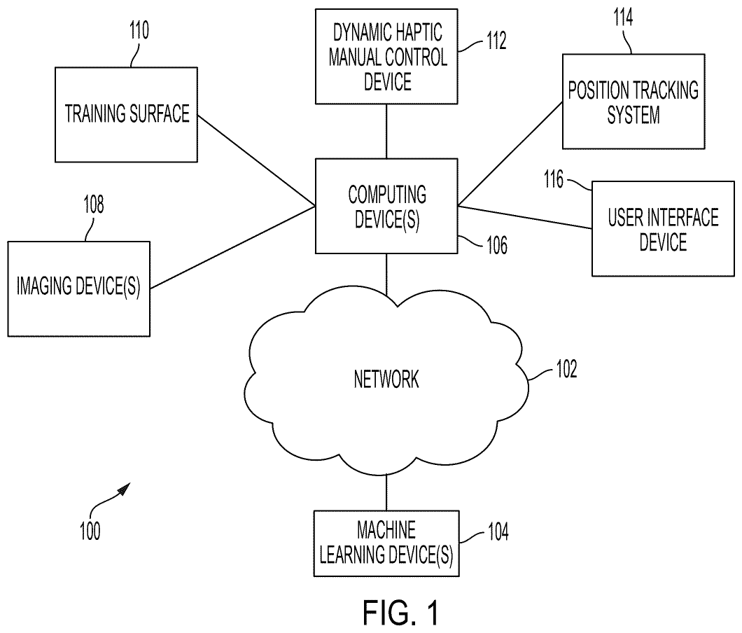

Referring now to the drawings, FIG. 1 depicts an illustrative automated training system 100 of networked devices and systems for carrying out methods that are used to train human users on how to perform certain procedures, particularly medical procedures such as CVC. In addition the components depicted in FIG. 1 can also be used to train components to provide user-facing functionality described herein. The automated training system 100 includes a network 102 that communicatively couples one or more machine learning devices 104 and one or more computing devices 106 such that data may be transmitted between the cone or more machine learning devices 104 and the one or more computing devices 106. The network 102 may be, for example, a wide area network (e.g., the internet), a local area network (LAN), a mobile communications network, a public service telephone network (PSTN) and/or other network and may be configured to electronically connect the one or more machine learning devices 104 and the one or more computing devices 106.

As also shown in FIG. 1, the automated training system 100 further includes one or more imaging devices 108, a training surface 110, a dynamic haptic manual control device 112, a position tracking system 114, and one or more interactive user interface devices 116. Each of the one or more imaging devices 108, the training surface 110, the dynamic haptic manual control device 112, and position tracking system 114, and the one or more interactive user interface devices 116 is communicatively coupled to the one or more computing devices 106, as indicated by the lines between objects. However, the present disclosure is not limited to such, and various components may be communicatively coupled to one another in an ad-hoc network, may be communicatively coupled to one another via the network 102, may be communicatively coupled via intermediary devices, and/or the like.

The one or more computing devices 106 may generally include hardware components particularly configured and arranged to carry out the various processes described herein. In some aspects, the one or more computing devices 106 may be physically attached to one or more components of the automated training system 100, integrated with one or more components of the automated training system 100, or the like. As noted, the one or more computing devices 106 may include various hardware components that allow the one or more computing devices 106 to carry out various processes described herein, such as, for example, processor circuitry, data storage devices (e.g., non-transitory, processor readable storage media), and/or the like. As used herein, “processor circuitry” is defined to include (i) one or more special purpose electrical circuits structured to perform specific operation(s) and including one or more semiconductor-based logic devices (e.g., electrical hardware implemented by one or more transistors), and/or (ii) one or more general purpose semiconductor-based electrical circuits programmable with instructions to perform specific operations and including one or more semiconductor-based logic devices (e.g., electrical hardware implemented by one or more transistors). Examples of processor circuitry include programmable microprocessors, Field Programmable Gate Arrays (FPGAs) that can instantiate instructions, Central Processor Units (CPUs), Graphics Processor Units (GPUs), Digital Signal Processors (DSPs), XPUs, or microcontrollers and integrated circuits such as Application Specific Integrated Circuits (ASICs). For example, an XPU can be implemented by a heterogeneous computing system including multiple types of processor circuitry (e.g., one or more FPGAs, one or more CPUs, one or more GPUs, one or more DSPs, etc., and/or a combination thereof) and application programming interface(s) (API(s)) that can assign computing task(s) to whichever one(s) of the multiple types of processor circuitry is/are best suited to execute the computing task(s).

The one or more imaging devices 108 are each generally any device that is capable of capturing images, video, or raw data of an area in its field of view, namely, the training surface 110, as will be described in greater detail herein. The one or more imaging devices 108 can capture images that include, for example, raw video and/or a 3D depth data stream (e.g., captured by a depth sensor such as a LIDAR sensor or the like). The one or more imaging devices 108 further include components that allow captured images, data, and/or the like to be transmitted (e.g., wirelessly or over a wired connection) to at least the one or more computing devices 106. As the various components of imaging devices 108 are generally understood, they are not discussed in greater detail herein.

The training surface 110 is generally a surface positioned within the field of view of the one or more imaging devices 108 that includes various components, such as sensors or the like (as described in further detail herein) that are communicatively coupled to the one or more computing devices 106 for transmitting data during a training procedure. The training surface 110 may further support a tray or the like thereon, which contains various tools or the like that are used for training, as well as a specialized portion utilized by a user to learn various procedures, as described in greater detail below.

The dynamic haptic manual control device 112 is generally a device that is used by a user during a training process, together with the various additional components described herein, to pierce a portion of the training surface 110. The dynamic haptic manual control device 112 includes feedback elements that provide the user with feedback during a procedure, as well as various sensors that are communicatively coupled to the one or more computing devices 106 for the transmission of data, signals, and/or the like. As will be described in greater detail herein, the dynamic haptic manual control device 112 also includes elements that allow a user to select particular settings that correspond to specific procedures, as well as components that simulate environmental conditions to the user during a training process. While the present disclosure is not limited to any particular device or components, for the purposes of clarity, the dynamic haptic manual control device 112 is a syringe device that includes elements for providing a user with feedback when inserted into the particular area of the training surface 110, as well as sensors for determining a location, positioning, arrangement, and movement of the dynamic haptic manual control device 112 for the purposes of training a user during a training process.

The position tracking system 114 is generally one or more devices that include sensors that are usable by a user in carrying out a training process and/or by the automated training system 100 in tracking various components described herein. In some embodiments, the position tracking system 114 may be an ultrasound probe or the like (e.g., a mock ultrasound probe). In other embodiments, the position tracking system 114 may include one or more sensors disposed on or around a simulated area, such as for example, a simulated subcutaneous area 206, within a funnel system 206b and/or a false vein 206c, as will be described in greater detail herein. In some aspects, the position tracking system 114 may include or incorporate sensors such as Hall effect sensors, or other electromagnetic based tracking devices and/or systems. The position tracking system 114 is communicably coupled to the one or more computing devices 106 for the purposes of transmitting data, signals and/or the like.

The one or more interactive user interface devices 116 are generally various hardware components that provide one or more user interfaces for communicating information to or from a user. For example, the one or more interactive user interface devices 116 may include any component that can receive inputs from a user and translate the inputs to signals and/or data that cause operation of the one or more computing devices 106 (e.g., a touchscreen interface, a keyboard, a mouse, and/or the like). In embodiments, the interactive user interface devices 116 can provide a user with a set of instructions for completing a procedure, provide visual, audio, and/or haptic feedback, and/or provide one or more interactive software programs to a user.

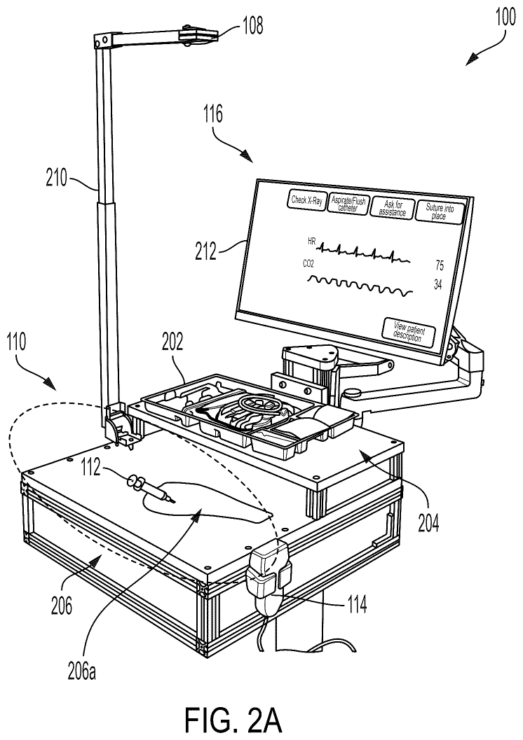

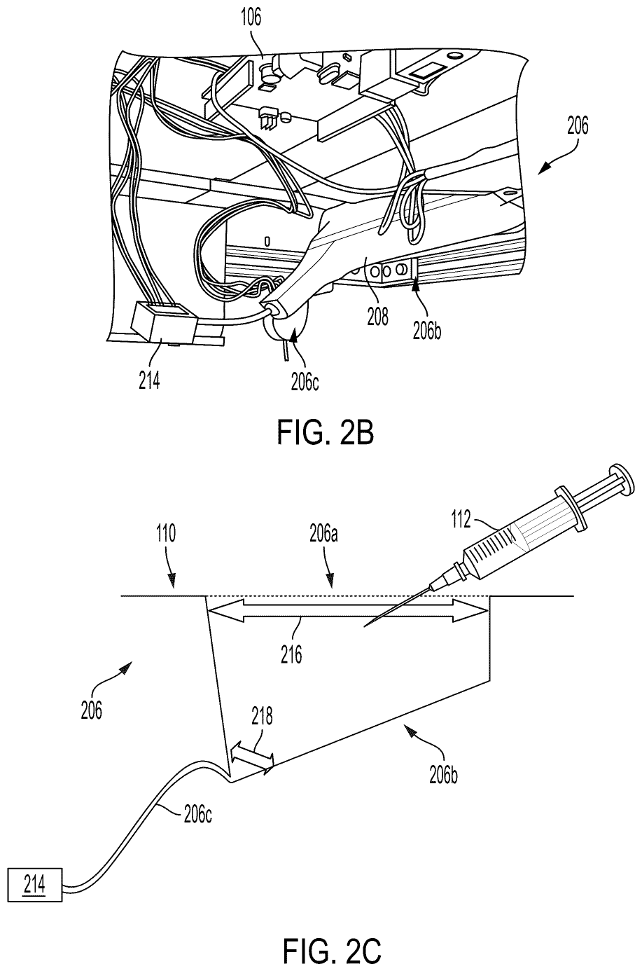

Turning now to FIG. 2A, one illustrative example the automated training system 100 is depicted. As shown in FIG. 2A, the one or more imaging devices 108 are positioned on a support 210 (e.g., a support arm or the like) over the training surface 110 and a tray 202 such that the training surface 110 and the tray 202 are within a field of view of the one or more imaging devices 108. The tray 202 is generally a container or the like that supports or includes one or more tools 204 therein, such as medical devices or the like that may be used during a training procedure. In some embodiments, the tools 204 may be actual tools that are utilized during a procedure, such as a medical procedure. In other embodiments, the tools 204 may be training tools that are designed and constructed to simulate actual tools, but are used for training purposes only. For example, the tools 204 may be blunted medical tools which may optionally be color coded or otherwise tagged with indicia or the like to aid in computer vision detection. That is, the tools 204 may be colored or otherwise include indicia thereon that allow the one or more computing devices 106 (FIG. 1) to recognize the tools 204 from image data received from the one or more imaging devices 108 by correlating the color, indicia, and/or other features of the tools 204 with stored data, such as a look up table or the like. Illustrative examples of the tools 204 include, but are not limited to, a guidewire, a catheter, a dilator, a scalpel, a syringe, or the like. It should be understood that the tools 204 may vary based on the particular procedure that is being trained.

The one or more interactive user interface devices 116 include a display 212, which is mounted adjacent to the training surface 110 such that menus, feedback, and other information is viewable by a user while using the training surface 110 (e.g., by operating the position tracking system 114 and/or the dynamic haptic manual control device 112 over or through a pierceable area 206a of the training surface 110, as described herein). In some implementations, the display 212 may be a touchscreen display, capable of receiving inputs from a user (e.g., menu selection, manipulation of images, other software interaction, etc.).

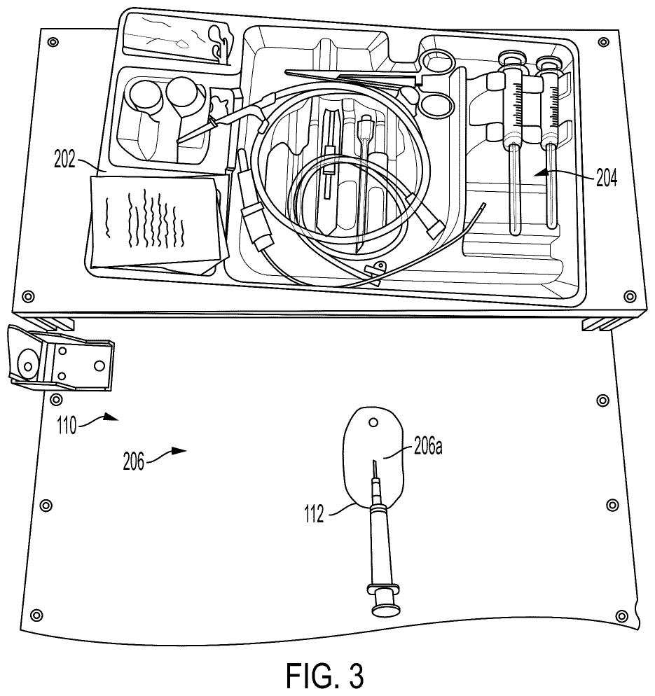

Referring to FIG. 2A, FIG. 2B, FIG. 2C, and FIG. 3 the training surface 110 includes a simulated subcutaneous area 206 that is accessible via the pierceable area 206a located in the training surface 110. The pierceable area 206a is generally a portion of the training surface 110 that is porous or semiporous such that objects (e.g., needles, blades, etc.) can be inserted therethrough to access a funnel system 206b and a false vein 206c disposed underneath the training surface 110. The pierceable area 206a, funnel system 206b, and false vein 206c together form the simulated subcutaneous area 206. The funnel system 206b is generally a cavity that is particularly shaped to guide objects that pierce the pierceable area 206a towards the false vein 206c. That is, the funnel system 206b funnels from a relatively larger surface area (the pierceable area 206a) to a relatively smaller surface area (the false vein 206c).

Said another way, and with reference specifically to FIG. 2C, the funnel system 206b includes one or more sidewalls 208 that narrow when traversed from the pierceable area 206a having a first diameter 216 to the false vein 206c having a second diameter 218, where the first diameter 216 is greater than the second diameter 218. As such, when an object is inserted through the pierceable area 206a, contact of the object with a sidewall 208 of the funnel system 206b acts to guide the object along the sidewall 208 towards the false vein 206c. One or more subcutaneous sensors 214 disposed in or around the simulated subcutaneous area 206 are particularly positioned and/or configured to sense the objects inserted therein, and data from the subcutaneous sensors 214 is transmitted to the one or more computing devices 106 for the purposes of determining a location, orientation, and position thereof, as described in greater detail herein. It should be appreciated that while FIG. 2B and FIG. 2C specifically depict a single subcutaneous sensor 214 coupled to the false vein 206c, the present disclosure is not limited to such a location or number.

The subcutaneous sensors 214 are generally any sensing hardware that can be used to determine the presence of an object, as well as positioning and/or orientation. For example, the subcutaneous sensors 214 may be an array of Hall effect sensors that are positioned along a length of the false vein 206c, the funnel system 206b, and/or the like. The array may interact with one or more magnets or other magnetic material disposed on the tools 204 and/or the dynamic haptic manual control device 112 and transmit signals to the computing devices 106 that are usable by the computing devices 106 to determine presence, positioning, and/or orientation. For example, the Hall effect sensors are placed with their centers 1 cm apart with the first sensor being 1 cm from the entrance to the false vein 206c. The distance from the magnet to each sensor is calculated from the measured voltage using the Equation (1):

$$d = \frac{ln ( \frac{V}{A R} )}{B} ( 1 ) \qquad ( 1 )$$

where d is the distance, V is the voltage, R is the resolution of the microcontroller, and A and B are experimentally determined constants. A and B are calculated by recording the voltage read by the computing devices 106 in five trials for individual hall effect sensors with the magnet at varying distances from the center of the sensor. Constants A and B for these sensors, with an R2 value of 0.86, are 104.07 and −0.447 respectively. The maximum distance these sensors can read with a magnet of this size was found to be 15 mm.

The difference in values from the dashed line and the experimental values at distances close to zero are mitigated by using measurements from multiple sensors as defined in Equation (2) below. The insertion distance along the cylindrical vessel is calculated by comparing the distances read by consecutive pairs of Hall effect sensors in the array. This is accomplished through the following conditional equation:

$$D = \{ \begin{matrix}{P}_{1} - {d}_{1} & only {d}_{1} > 0 \\ \frac{( {P}_{n} - {d}_{n} ) + ( {P}_{n - 1} - {d}_{n - 1} )}{2} {d}_{n} , & {d}_{n - 1} > 0 \\ {P}_{8} + {d}_{8} & only {d}_{8} > 0\end{matrix} \qquad ( 2 )$$

where D is the insertion distance, Pₙ is the position of the nth sensor in the array, and dₙ is the distance read by the nth sensor as defined in Equation (1). The Hall effect array was experimentally evaluated using a 12 cm piece of 7.5 mm diameter transparent plastic tubing, which was mounted over a breadboard with 8 hall effect sensors in an array. Markings were drawn on the catheter every 5 mm starting from the location of the magnet. The experiment was conducted in two tests, one static and one dynamic. In the static test, the catheter was inserted and held in place while 30 measurements were taken at 10 Hz. This was done at 5 mm increments from a position of 0 to 85 mm. In the dynamic test, the catheter was continuously inserted the full 85 mm at a rate of 5 mm/s while measurements were recorded at 10 Hz. Each test was repeated 5 times.

In addition to Hall-effect sensors, the subcutaneous sensors 214 described herein may also include, in the alternative or addition, optical sensors, light sensors, mechanical switches, potentiometers, optical position sensors, ultrasonic sensors, laser sensors, linear variable differential transformers, and/or the like. As noted herein, the subcutaneous sensors 214 may be particularly positioned and/or arranged to detect and provide data pertaining to the various items that may be inserted into the simulated subcutaneous area 206, including identification, location, positioning, movement, and/or the like. As such, the particular positioning and/or arrangement of the subcutaneous sensors 214 within the simulated subcutaneous area 206 may be based on the type of sensor used, the number of sensors used, and/or the types of objects to be detected and sensed.

Referring to FIG. 3, when in use, a user may access the various tools 204 on the tray 202 to perform a simulated procedure on the simulated subcutaneous area 206 of the training surface 110 using the tools 204, the dynamic haptic manual control device 112, and/or the position tracking system 114 (FIG. 2A) as described herein. The location of the tray 202 adjacent to the training surface 110 provides an all-in-one area for training a user with items for training in an easy to access space. In addition, both the tray 202 and the training surface 110, in being proximally located with respect to one another, can be imaged in the same field of view of the one or more imaging devices 108 (FIG. 2A).

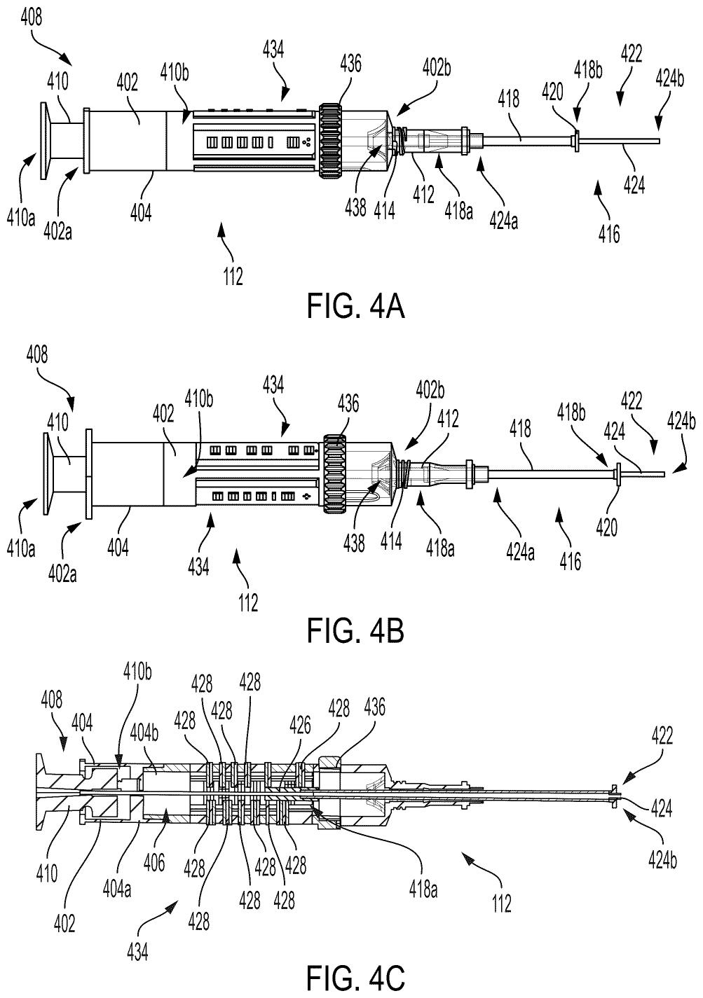

Turning now to FIG. 4A, FIG. 4C, and FIG. 4C, an illustrative example of the dynamic haptic manual control device 112 is depicted. As noted above, the particular example of the dynamic haptic manual control device 112 described herein is a syringe that is used to train a user on processes for a CVC procedure. However, it should be appreciated that the dynamic haptic manual control device 112 encompasses other devices that also are manually manipulated by a user and provide haptic feedback according to training protocol are contemplated and included within the scope of the present disclosure.

FIG. 4A and FIG. 4B depict side views of the dynamic haptic manual control device 112 (FIG. 4B is shown with the dynamic haptic manual control device 112 rotated along a longitudinal axis approximately 90 degrees). As shown in FIG. 4A and FIG. 4B, the dynamic haptic manual control device 112 generally includes a hollow syringe body 402, a syringe plunger 408, a hub 412, and a telescopic needle assembly 416. Referring also to FIG. 4C, the hollow syringe body 402 is generally elongate and includes one or more sidewalls 404 that define a cavity 406. The one or more sidewalls 404 include an exterior surface 404a and an interior surface 404b. Referring again to FIG. 4A and FIG. 4B, the hollow syringe body 402 also includes a proximal end 402a and a distal end 402b opposite the proximal end 402a. The proximal end 402a is open to receive the syringe plunger 408 therein. As such, the opening at the proximal end 402a is generally shaped and sized to correspond to a shape and size of the syringe plunger 408. In some aspects, the distal end 402b is also open and can receive portions of the telescopic needle assembly 416 as described in greater detail below.

The syringe plunger 408 generally includes a plunger body 410 that defines a proximal end 410a and a distal end 410b. The distal end 410b is generally shaped and sized to be inserted within the open proximal end 402a of the hollow syringe body 402 into the cavity 406. As will be described in additional detail herein, the distal end 410b may include one or more features (e.g., surface features, etc.) for interacting with various components inside the hollow syringe body 402. In some aspects the proximal end 410a may include a surface, a grip, a ring feature, and/or the like that facilitates manipulation of the syringe plunger 408 by a user such that the syringe plunger 408 can be pushed or otherwise directed distally into the cavity 406 of the hollow syringe body 402 and/or pulled or otherwise retracted proximally from the hollow syringe body 402. It should be appreciated that moving the syringe plunger 408 distally decreases a volume of a cavity of the hollow syringe body 402 (e.g., cavity 406 or a proximal plunger cavity that is separate from cavity 406) and moving the syringe plunger 408 proximally increases the volume of a cavity of the hollow syringe body 402. In some aspects, the syringe plunger 408 may also include a bore therethrough that is configured to receive additional components at the distal end 410b, such as a guidewire or the like. As will be discussed herein, the syringe plunger 408 is generally configured to simulate an aspiration process by providing tactile feedback to a user operating the syringe plunger 408.

The hub 412 is generally a supporting device for removably coupling the telescopic needle assembly 416 to the distal end 402b of the hollow syringe body 402. For example, as depicted in FIG. 4A and FIG. 4B, the hub 412 may include a mating feature 414 that mates with the distal end 402b of the hollow syringe body 402. For example, the mating feature 414 may be threads, a luer lock, and/or the like. As such, the hub 412 is removably coupled to the hollow syringe body 402. This removable coupling may be useful during a simulated procedure where a user utilizes the hollow syringe body 402 and the syringe plunger 408, then decouples the hollow syringe body 402 from the hub 412 for the purposes of inserting other items from the set of tools 204 (FIG. 3), such as a guidewire, a catheter, a dilator, or the like.

In some aspects, the hollow syringe body 402 may further include one or more LEDs 438 disposed within the cavity 406 and/or on an exterior surface 404a of the sidewalls 404. The LEDs 438 are generally actuable during an aspiration process to illuminate when particular signals are received from the computing devices 106 (FIG. 1) to indicate simulated blood draw to a user. That is, if the computing devices 106 determine from various signals that a user is completing an aspiration process in a predetermined manner, the computing devices 106 may transmit one or more signals and/or provide electrical power to the LEDs 438 to illuminate. It should be appreciated that the LEDs 438 are merely illustrative, and other devices or components that can be actuated to indicate blood draw are also contemplated and included within the scope of the present disclosure.

Still referring to FIG. 4A and FIG. 4B the telescopic needle assembly 416 includes a hollow outer needle 418 and an inner needle 422. The hollow outer needle 418 is elongate with a proximal end 418a and a distal end 418b spaced apart from the proximal end 418a. The hollow outer needle 418 generally has a length from the proximal end 418a to the distal end 418b that is sufficient to extend into the cavity 406 of the hollow syringe body 402. This is because the proximal end 418a of the hollow outer needle 418 includes an end effector 426 thereon that engages with slip flexures 428, as described herein. As such, the length of the hollow outer needle 418 is sufficient for the end effector 426 to engage with the slip flexures 428 inside the cavity 406 of the hollow syringe body 402. The end effector 426 is generally a protrusion extending radially outwards from the hollow outer needle 418. In some aspects, the end effector 426 is constructed of a generally rigid material, such as, for example, stainless steel or a rigid polymer material. In some aspects, the end effector 426 may be formed on a surface of the hollow outer needle 418 (e.g., via a deposition process, overmolding, fixing, or the like). In other aspects, the end effector 426 may be integral with the body of the hollow outer needle 418 (e.g., the hollow outer needle 418 is formed with additional material at the proximal end 418a thereof that extends radially outward). The size of the end effector 426 is generally not limited by the present disclosure so long as the dimensions of the end effector 426 allow for contact with slip flexures 428, as described in greater detail herein. For example, the end effector 426 may extend a particular distance radially outward such that the end effector 426 can be contacted with components such as slip flexures 428 disposed within the hollow syringe body 402. A length of the end effector 426 may generally be any length, and is not limited by the present disclosure. For example, in some embodiments, the end effector 426 may extend a length that is less than a total length of the hollow outer needle 418. In other embodiments, the end effector 426 may extend an entire length of the hollow outer needle 418. Like the size, the shape of the end effector 426 is generally not limited by the present disclosure, so long as the shape allows for engagement with only particular ones of the slip flexures 428 at a time while other ones of the slip flexures 428 are not engaged, as described in greater detail herein. For example, and briefly referring to FIG. 6, the end effector 426 is shaped such that it protrudes radially outward from a portion of the hollow outer needle 418 while other portions of the hollow outer needle 418 do not include such a protrusion. For example, when traversing a circumference of the hollow outer needle 418, about one fourth (25%) of the circumference of the hollow outer needle 418 contains the protrusion of the end effector 426, while the remainder of the of the circumference of the hollow outer needle 418 (e.g., about three fourths or 75%) does not contain the protrusion. In some aspects, as shown in FIG. 6 for example, the end effector 426 protruding from the hollow outer needle 418 may have a rounded triangle or teardrop shape. However, other shapes are contemplated and included within the scope of the present disclosure. In some embodiments, the end effector 426 may be particularly positioned with respect to the various other components of the hollow syringe body 402 (e.g., the slip flexures 428) to ensure engagement as described herein. For example, the portion of the hollow outer needle 418 containing the end effector 426 may face a particular direction (e.g., downward in FIG. 6) to ensure engagement. Such a positioning may be fixed or adjustable (e.g., adjusted to engage with certain ones of the slip flexures 428 as described herein).

Referring again to FIGS. 4A-4B, the proximal end 418a of the hollow outer needle 418 is movable within the hub 412 so as to extend through the hub 412 into the cavity 406 of the hollow syringe body 402. In some aspects, the hollow outer needle 418 may also include a needle cap 420 at the distal end 418b that is flared radially outwards from the body of the hollow outer needle 418. For example, the needle cap 420 may be a flange or the like at the distal end 418b of the hollow outer needle 418 that acts as a stop, preventing a user from inserting the distal end 418b of the hollow outer needle 418 into the funnel system 206b of the simulated subcutaneous area 206 (FIG. 2C), but rather causes the proximal end 418a of the hollow outer needle 418 to move proximally into the cavity 406 of the hollow syringe body 402. While not depicted in FIGS. 4A-4B, in some embodiments, the hollow outer needle 418 may be selectively engaged with a biasing assembly or the like that biases the hollow outer needle 418 distally (e.g., to return the hollow outer needle 418 to an initial position after use as described herein). Such a biasing assembly may be selectively engaged with the hollow outer needle 418 in order to avoid the biasing assembly from affecting the feedback profile of the slip flexures 428 as described herein. In some embodiments, the hollow outer needle 418 may be manually maneuvered in a distal direction after use to return the hollow outer needle 418 to an initial position.

Referring also to FIG. 2C and FIG. 4C, the inner needle 422 includes an elongate body 424 having a proximal end 424a and a distal end 424b spaced a distance from the proximal end 424a. The proximal end 424a is generally fixed to the hub 412 and does not extend or retract. Instead, the hollow outer needle 418 is movable along the length of the elongate body 424 of the inner needle 422 as described herein. The elongate body 424 is generally sized such that a length from the proximal end 424a to the distal end 424b is sufficient to access the false vein 206c when the inner needle 422 pierces the pierceable area 206a and is inserted into the funnel system 206b.

As specifically shown in FIG. 4C, but with reference also to FIGS. 4A-4B, the one or more sidewalls 404 of the hollow syringe body 402 include a plurality of haptic cartridges 434 that each include a slip flexure 428 that extends radially inwards from the sidewalls 404 into the cavity 406 of the hollow syringe body 402. In some aspects, the haptic cartridges 434 generally extend through the sidewalls 404 of the hollow syringe body 402 such that the slip flexures 428 extend internally from the interior surface 404b of the sidewalls 404. In other aspects, the haptic cartridges 434 may be split such that a portion of each haptic cartridge 434 is located on the exterior surface 404a of the sidewalls 404 and a corresponding portion of the haptic cartridge 434 (in the form of the slip flexure 428) is located, fixed, positioned, or otherwise integrated with the interior surface 404b of the sidewalls 404 (e.g., via overmolding, insert molding, forming as a singular piece, etc.). The slip flexures 428 are generally compliant elements that can be moved in and out of engagement with the end effector 426 disposed on the hollow outer needle 418 when the hollow outer needle 418 is advanced proximally into the cavity 406 of the hollow syringe body 402. While the present disclosure is not limited to any particular number of haptic cartridges 434 (and corresponding slip flexure 428) within the cavity 406 of the hollow syringe body 402, the number of haptic cartridge 434 and slip flexures 428 generally corresponds to a number of haptic profiles for the dynamic haptic manual control device 112 that can be selected by a user via a selection ring 436 as described herein.

The slip flexures 428 are generally configured to generate realistic haptic profiles in cartridges, which can be rotated by the selection ring 436 to enable dynamic haptic feedback. The slip flexures 428 leverage compliance and controlled friction to develop negative slopes in force-displacement curves and enable the creation of haptic compliant mechanisms capable of infinite displacement range. The slip flexures 428 have the potential to improve haptic simulation systems, but could also be used in many industries to improve compliant mechanism designs that are currently limited in range. As noted, the slip flexures 428 are compliant elements that leverage compliance and controlled friction to generate specific haptic curves by varying the topology, geometry, and material of the slip flexures 428. Referring also to FIG. 7 in addition to FIG. 4C, the slip flexures 428 are shown in step 1 in an initial configuration. The slip flexures 428 are designed to activate when the end effector 426 is pressed against a radially innermost edge of the slip flexure 428 (step 2) and flexes proximally, producing a resisting force that increases with displacement (step 3). This resisting force increases until the slip flexure 428 deforms enough to slip off the end effector 426, introducing a lower resisting force due to friction. This friction force is active until the end effector 426 has passed the slip flexure 428 at which time the resisting force returns to zero (step 4). Due to the controlled slippage in the design, compliant mechanisms utilizing the slip flexure 428 are capable of infinite range, an aspect of compliant mechanisms that has been severely limited in previous designs.

The topology of each slip flexure 428 is determined by the selection and placement of various layers of the slip flexure 428, the geometry indicates the shape of the individual slip flexures, and the material stiffness is varied to produce various amplitudes of force reactions. By varying these three aspects of the design, the desired haptic profiles can be generated. Illustrative examples of geometries of slip flexures 428 are depicted in FIG. 8. More specifically, FIG. 8 depicts a cross sectional view showing eight different geometries (a-h) of slip flexures 428, each mounted to a mounting tab 702 that is coupled to or integrated with the sidewalls 404 of the hollow syringe body 402 (FIG. 4C) as described herein. Referring to FIG. 8, illustrative geometries of slip flexure 428 include, but are not limited to, a first rectangular geometry (a) that has two sides longer than a second rectangular geometry (b). As such, the slip flexure 428 in the first rectangular geometry (a) would provide more engagement and resisting force to the end effector 426 because it would take the end effector 426 longer to traverse the engagement with the slip flexure 428. In addition, the slip flexures 428 in geometries (c), (d), (e), and (f) are generally trapezoidal geometries. However, the lengths of each of the sides of the trapezoids varies in each geometry, which results in different characteristics of engagement with the end effector 426. For example, geometries with side walls having a relatively shallow slope (e.g., the walls extending radially from the mounting tab 702, such as geometries (c), (e), and (f)) cause a more gradual slope of engagement from when the slip flexure 428 first engages with the end effector 426 until it reaches the apex (e.g., the lateral side of the trapezoidal geometry) relative to the geometries having a relatively steeper slope (e.g., geometry (d)). In another example, the slip flexures 428 in geometries (g) and (h) are reverse trapezoidal, thereby providing a different type of feedback that is sharper as the engagement with the slip flexure 428 with the end effector 426 transitions from the side walls to the lateral side of the geometries relative to the trapezoidal geometries. As will be appreciated, the various geometries of FIG. 8 each cause unique feedback to the user as a result of engagement of the end effector 426 with the slip flexure 428, which can be used to simulate real-world scenarios for various anatomies or procedures during insertion. In addition, it should be understood that the geometries depicted in FIG. 8 are merely illustrative and other geometries that cause different feedback profiles not specifically shown herein are contemplated and included in the scope of the present disclosure. Table 1 below depicts illustrative measured characteristics of engagement with the various slip flexures 428 with the end effector 426 under experimental conditions:

| | Slope | Peak | Slip Point | Friction | Slip Point |
|---|---|---|---|---|---|
| Geometry | (N/mm) | Force (N) | 1 (mm) | Force (N) | 2 (mm) |
| (a) Straight | 0.056 | 0.106 | 1.9 | 0.022 | 5.2 |
| (b) Straight | 0.046 | 0.060 | 1.3 | 0.013 | 5.0 |
| (c) Trapezoid | 0.050 | 0.066 | 1.3 | 0.010 | 4.7 |
| (d) Trapezoid | 0.045 | 0.077 | 1.7 | 0.021 | 5.6 |
| (e) Trapezoid | 0.041 | 0.061 | 1.5 | 0.007 | 5.0 |
| (f) Trapezoid | 0.038 | 0.050 | 1.3 | 0.007 | 5.5 |
| (g) Inverted | 0.036 | 0.051 | 1.4 | 0.006 | 5.0 |
| Trapezoid | | | | | |
| (h) Inverted | 0.025 | 0.034 | 1.4 | 0.000 | 5.5 |
| Trapezoid | | | | | |

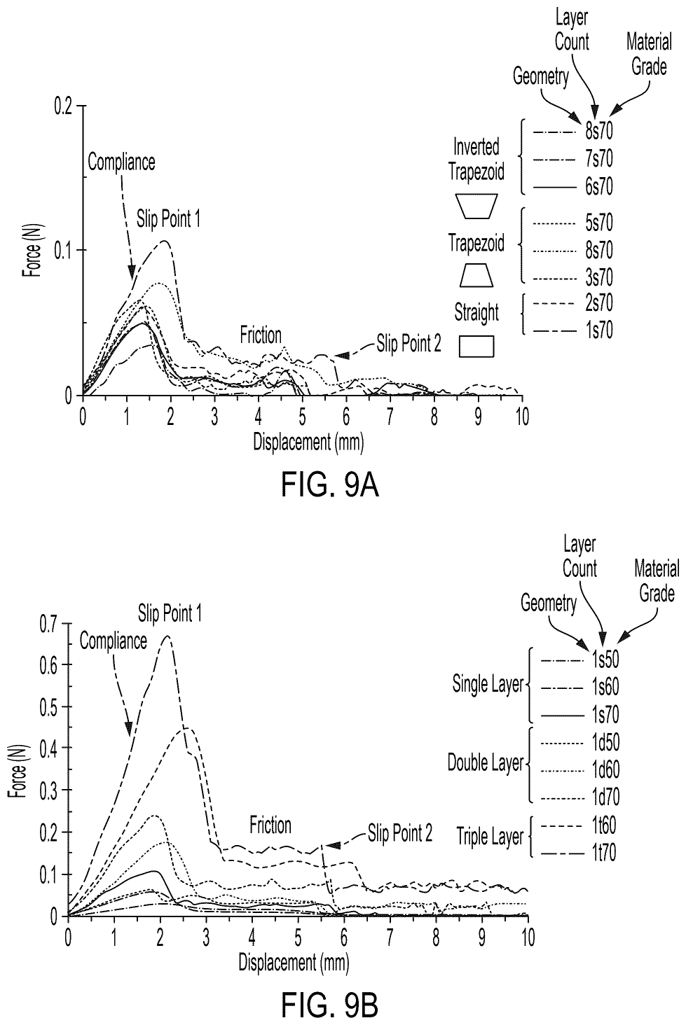

The haptic curve features varied based on the geometry, material, and layer count. As shown in Table 1 above, Geometry (a) exhibited the highest peak force, slope, slip point 1 displacement, and friction. Varying the geometry of the slip flexure 428 changes the magnitude of the peak without drastically altering the slope or the friction, as depicted in FIG. 9 in graph (a). On the contrary, varying the material hardness and layer count, as shown in FIG. 9 in graph (b) changes the slope and magnitude of the haptic profile, while also affecting the friction after the initial slip displacement. Overall, these results confirm that by modifying the geometry, material, and layer count of the slip flexures 428, a variety of haptic profiles can be produced where each haptic profile contains the core features.

Referring again to FIGS. 4A-4C, the material for the slip flexures 428 is not limited by the present disclosure, and can generally be any material that is pliant and can provide the frictional engagement with the end effector 426 as described herein. The material can be a single block of material, or may be successive layers of the same or varying materials to achieve a particular profile for the slip flexure 428. One illustrative example of a material is a 1/32 inch thick silicone rubber. In some aspects, the material selected for each slip flexure 428 may have a particular material hardness grade, such as, for example, grade 50, grade 60, or grade 70.

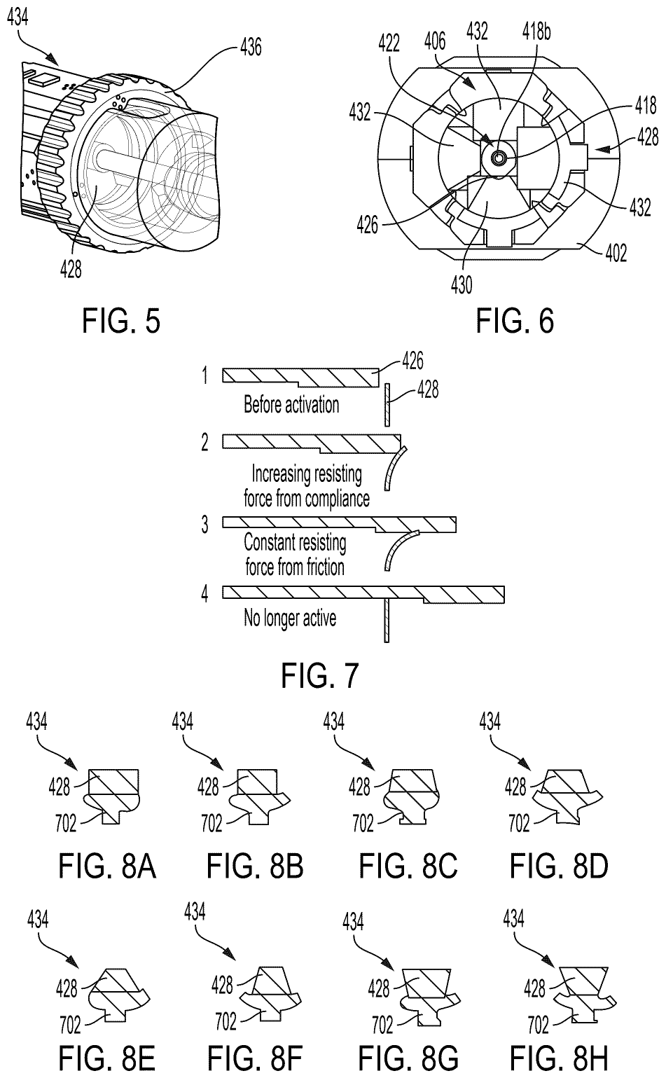

As noted, the hollow syringe body 402 may be formed with a plurality of haptic cartridges 434 and corresponding slip flexures 428 that extend from the interior surface 404b of the sidewalls 404 into the cavity 406 thereof. Since each slip flexure 428 is formed to produce a different feedback profile, the hollow syringe body 402 is further structured so that a user can selectively actuate a particular haptic cartridge 434 (and corresponding slip flexure 428) for use. For example, a user can selectively actuate a particular haptic cartridge 434 that corresponds to various anatomical profiles that account for factors such as skin thickness, adipose tissue depth, or the like. Accordingly, as depicted in FIGS. 4A-4C, and with reference to FIG. 5, the hollow syringe body 402 further includes the selection ring 436 disposed on the exterior surface 404a of the sidewalls 404. The selection ring 436 is engageable with each haptic cartridge 434 so as to move the corresponding slip flexure 428 between active and inactive states. In some embodiments, the selection ring 436 may be mechanically coupled to all of the haptic cartridges 434 such that rotation of the selection ring 436 causes rotation of all of the haptic cartridges 434 together, with positioning of the haptic cartridges 434 and corresponding slip flexures 428 with respect to the end effector 426 determining which slip flexures are active and which are inactive, as described below. In other embodiments, the selection ring 436 may include one or more mechanical linkages (e.g., a drive shaft and a transmission) that allows for selective coupling to each of the haptic cartridges 434 independently. In some aspects, the selection ring 436 may include indicia thereon that indicates which slip flexures 428 are located in particular positions with respect to the selection ring 436 so as to provide a user with a means of determining which slip flexures 428 are active and inactive. It should be appreciated that the selection ring 436 is only one illustrative example of a component that allows for selective engagement of certain haptic cartridges 434 and/or slip flexures 428, and other mechanisms are contemplated and included within the scope of the present disclosure. For example, each of the haptic cartridges 434 and corresponding slip flexures 428 may be removable from the hollow syringe body 402 and replaced with other haptic cartridges and corresponding slip flexures 428 having different profiles. In another example, each of the haptic cartridges 434 and corresponding slip flexures 428 may be biased outwardly when not in an active state, but can be actuated (e.g., by applying a force that overcomes the biasing assembly, by actuating a mechanical device, by actuating an electronically controlled device, etc.) to place in an active state. In still another example, a sliding mechanism may be utilized to selectively slide each haptic cartridge 434 and corresponding slip flexures 428 into or out of an active state.

Engagement of the slip flexures 428 with the end effector 426 is particularly depicted in FIG. 6, which shows a cross sectional view of the cavity 406 of the hollow syringe body 402. As shown in FIG. 6, the hollow outer needle 418 is disposed centrally within the cavity 406 of the hollow syringe body 402 with the end effector 426 extending in a particular direction radially outwards from the distal end 424b of the elongate body 424 (in FIG. 6, the end effector 426 extends downward, but this is merely illustrative). The various slip flexures 428 are disposed radially around the hollow outer needle 418. As particularly shown in FIG. 6, four slip flexures 428 are depicted, but as previously discussed, the number of slip flexures 428 is not limited by the present disclosure. Because of the dimensions of the elongate body 424 and the end effector 426 extending therefrom, only one of the slip flexure 428 contacts the end effector 426 and engages with the end effector 426 when the hollow outer needle 418 is positioned within the cavity 406 hollow syringe body 402. The particular slip flexure 428 contacting the end effector 426 may be referenced as an active slip flexure 430 while the other slip flexures 428 that are not contacting the end effector 426 may be referenced as inactive slip flexures 432. Manipulation of the selection ring 436 (FIG. 5) can move any one of the slip flexures 428 into contact with the end effector 426, thereby causing the slip flexure 428 to be the active slip flexure 430 at that particular moment.

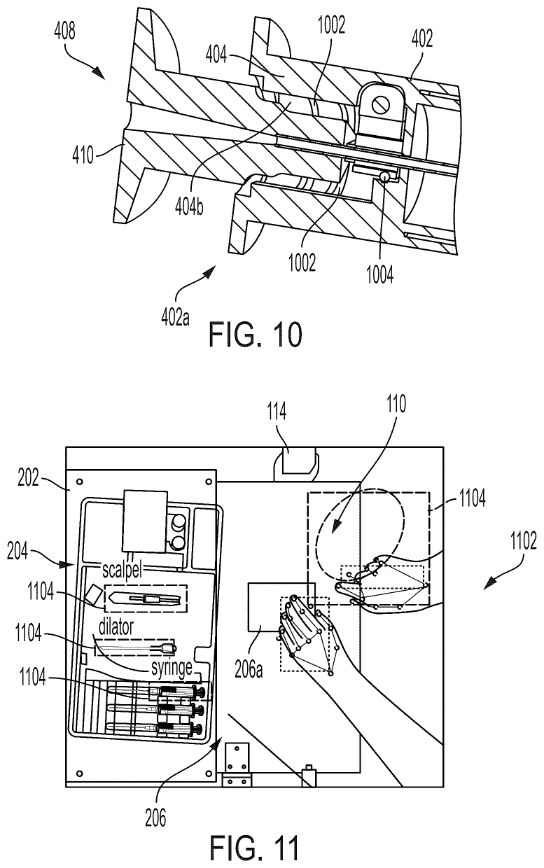

Referring now to FIG. 10, the proximal end 402a of the hollow syringe body 402 is depicted in cross section. As shown in FIG. 10 the interior surface 404b of the sidewalls 404 also includes one or more compliant mechanisms 1002 disposed thereon. These compliant mechanisms 1002 are generally positioned to engage with the plunger body 410 to provide resistance when the plunger body 410 is moved distally or proximally within the hollow syringe body 402. In addition, these compliant mechanisms 1002 may be shaped, sized, and/or disposed on the interior surface 404b of the sidewalls 404 in a particular manner so as to provide a particular feedback profile to a user when the user manipulates the plunger body 410 to move distally or proximally to mimic a real life procedure. For example, during an aspiration process whereby a user may manipulate the plunger body 410 to cause proximal movement of the plunger body 410, the compliant mechanisms 1002 engage with a portion of the plunger body to provide resistance that mimics real world conditions a user might experience. In various embodiments, the compliant mechanisms 1002 may be formed on the interior surface 404b, integrated with the interior surface 404b, or affixed to the interior surface 404b. The compliant mechanisms 1002 may be formed from the same material as the sidewalls 404 of the hollow syringe body 402, or may be formed from a different material. For example the compliant mechanisms 1002 may be formed from a polymer based material, steel, and/or the like.

Also depicted in FIG. 10 is a detector switch 1004 disposed within the hollow syringe body 402. The detector switch is generally positioned adjacent to the plunger body 410 so as to detect movement of the plunger body 410 within the hollow syringe body 402. Data from the detector switch 1004 is usable to determine how much the plunger body 410 is moved with respect to the hollow syringe body 402, which can then be used to provide feedback regarding a particular procedure. For example, when used for an aspiration procedure whereby the plunger body 410 would be moved proximally with respect to the hollow syringe body 402, the detector switch 1004 can provide data relating to a distance traversed in the distal direction, which in turn can be used to provide feedback on the simulated aspiration (e.g., by providing an indicator such light illumination, information on the display, etc.). The detector switch 1004 may be any switch or sensor, such as a mechanical contact switch, an optical sensor, a pressure sensor, or the like.

Generally referring to FIGS. 1-10, in operation, as the telescopic needle assembly 416 (particularly the inner needle 422 thereof) is inserted into the pierceable area 206a of the simulated subcutaneous area 206 on the training surface 110, the inner needle 422 penetrates the training surface 110 while the hollow outer needle 418 retracts into the hollow syringe body 402 and engages with one or more of the active slip flexures 428 within to produce the selected haptic profile. It should be appreciated that engagement with the slip flexures 428 may be a single slip flexure 428 or a plurality of successive slip flexures 428 (when traversing from a distal to a proximal direction). The user can select the haptic profile by rotating the selection ring 436 which rotates the various haptic cartridges 434. These haptic cartridge 434 each produce the haptic profile of a different anatomy. The tear-drop shaped end effector 426 of the hollow outer needle 418 is designed to only activate the slip flexures 428 of the selected profile (e.g., the active slip flexure 430) while sliding past the others (e.g., the inactive slip flexures 432). The compliant mechanism 1002 is designed to replicate the force felt when aspirating a real syringe (e.g., by engaging the compliant mechanism 1002 with the plunger body 410), and the detector switch 1004 provides specific data pertaining to movement of the plunger body 410. Additionally, the LEDs 438 are coupled to the hollow syringe body 402 to simulate blood draw during venous or arterial access. Upon successful insertion into a tissue pad, a guidewire can be passed through the syringe from the distal end of the plunger body 410 through the inner needle 422 into the simulated subcutaneous area 206 to trigger the subcutaneous sensors 214. Alternatively, the hollow syringe body 402 can be decoupled from the hub 412 so the guidewire can be inserted via the hub 412 through the inner needle 422 into the simulated subcutaneous area 206 to trigger subcutaneous sensors 214.

As noted herein, various sensors are utilized for the purposes of tracking a location, position, and orientation of various components of the automated training system 100, such as the tools 204 and/or the dynamic haptic manual control device 112, as well as various portions thereof. For example, the subcutaneous sensors 214 can determine presence, location, and orientation of devices as described herein. In addition, the imaging devices 108 can capture images that are used, via optical recognition and/or trained machine learning algorithms (e.g., those stored on machine learning devices 104) to recognize an object being used, determine the positioning of that object with respect to other objects. Together with the data from the subcutaneous sensor 214, the computing devices 106 can accurately observe and determine the use of objects during a simulated procedure and provide feedback to a user accordingly.

One such illustrative user interface that is generated as a result of imaging objects, identifying the objects, and determining positioning or the like is depicted in FIG. 11. FIG. 11 generally shows the various components previously discussed herein as imaged by the imaging devices 108. As shown in FIG. 11, the tray 202 includes the tools 204 thereon, as well as the training surface 110 and the simulated subcutaneous area 206 with the pierceable area 206a, the position tracking system 114. Various objects have been recognized using computer vision software and are bounded by boxes as a means of tagging. For example, FIG. 11 depicts a plurality of tagged tools 1104 bounded by boxes. In addition, the hands of a user 1102 have been recognized using computer vision software and are bounded by different boxes as a means of tagging. Various image recognition software can be utilized to independently track location, positioning, and movement of each of the tools, as well as location, positioning, and movement of a user's hands when manipulating or using the tools. In addition, the software is able to combine the independent tracking so as to estimate a location, positioning, and movement of the tools when obscured from view in the images (e.g., when a user is holding one of the tools and the user's hands obscures at least a portion of the tools from view in the images). In addition, the software can also estimate various obscured endoscopic component movement based on external manipulation of such tools (e.g., when a user rotates a knob of an endoscope).

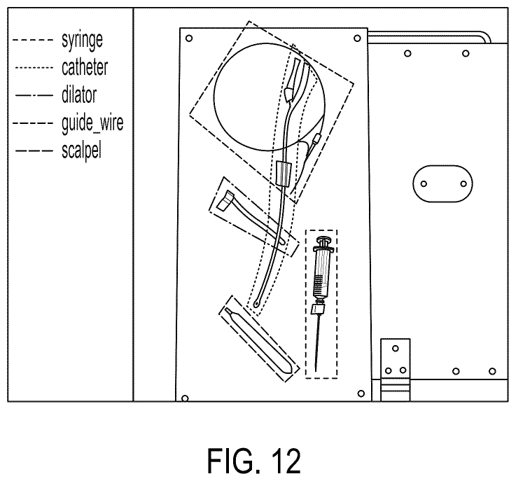

The algorithm which was deployed into the system is an open-source code name YOLOv5. The coding process is thus: The environment of YOLOv5 is established, then the modules are imported. The training parameters are set, such as bench or epochs. The training data is imported into python. In order to run YOLOv5 to train the machine learning (ML) model, the labeling is completed before the actual training occurs. Online ML tools by Roboflow (Des Moines, IA) were used to create the labels for the ML data set. Roboflow is a web-based service to create labeling or even training for machine learning models. FIG. 12 gives an example of a labeled input image.

To train the ML model, the YOLOv5 algorithm reads in the labeled data and, based on the characteristics of each image, builds the image detection algorithm. After the system finishes training the algorithm, it forms a file which can then be called as a function in python. During the development, the training data can be considered the most direct influence of the results toward the ML output. Three different algorithms were developed based on the number of training images in the data sets, from 100, 300, and 800. The training data was captured through the imaging devices 108 (FIG. 2A) and labeled with Roboflow. The first 100 training sets contain the medical tools distributed randomly on top of the training surface 110. A similar method was used to expand the database from 100 to 300 for the second ML algorithm. The final set was created based on these 300 images, using an image augmentation method, provided in Roboflow, to expand the data into 800 images. This augmentation method includes image rotation, light exposure rate, or mirroring the image. After the training process was complete, the validation data was passed through the system to test the accuracy and robustness of the system. Fifty validation images were taken in four different conditions to ensure the consistency of the algorithm. These validation images were collected by the same method as the training data, with the tools randomly placed on top of the tray. The only difference between the two data sets is the different environmental conditions, which can help validate the system under different circumstances. Two metrics, the precision rate and the recall rate, were assessed to determine accuracy of the machine learning model. The precision rate equation in machine learning code is calculated in Equation (3):

$$PR = \frac{TP}{TP + FP} \qquad ( 3 )$$

where PR is the precision rate, TP is the count of true positives, and FP is the count of false positives. A true positive is defined as an object which was detected in the image and was actually there. A false positive is an object that was detected in the image but was not actually there. The recall rate is calculated in Equation (4):

$$RR = \frac{TP}{TP + FN} \qquad ( 4 )$$

where RR is the Recall Rate, TP is the count of true positives, and FN is the count of false negatives. A false negative is defined as an object which was in the image but was not detected by the algorithm.

The current ML system provides an overall precision rate of 90.9% with the recall rate of 81.69%. One way to increase the system accuracy is to average out the response from the system. Those 50 sets of validation data are based on individual images instead of a live recording. When the system is running in real time, it would be possible to average the results across video frames. This will help the system automatically eliminate the outliers, thus increasing the accuracy. Furthermore, accuracy could be improved by further increasing the number of images in the set. This ML algorithm is usable to recognize the shape and/or the color of the tools.

The ML system can further be trained via other methodologies according to the present disclosure. For example, a user may interact with one of the one or more tools 204 while artificial intelligence is used to measure and interpret the interaction. Specifically, the automated training system 100 allows for the imaging devices 108 to record video to measure manipulation of tools (e.g., measure endoscopic knob rotation angle in real time). For example, a visual angle indicator attached underneath a tool may provide verified angle for an experiment, and experiments may be performed in a variety of trials, where in each trial, the tool is rotated in a stepped fashion (e.g., in various degree increments in a range of degrees such as, for example, 0°-10°, 0°-20°, 0°-30°, and so on). Aspects further relate to producing labels from ultraviolet (UV) light to gather training data for ML to identify the location of medical tools.

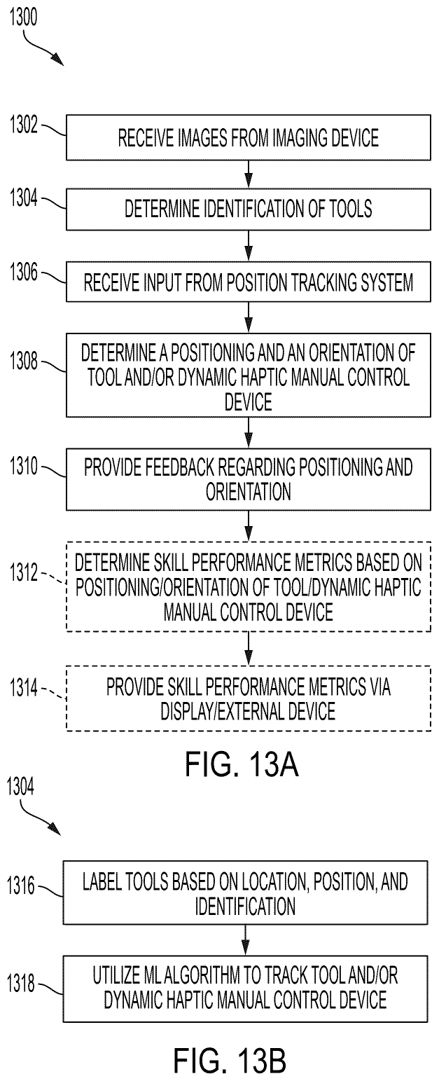

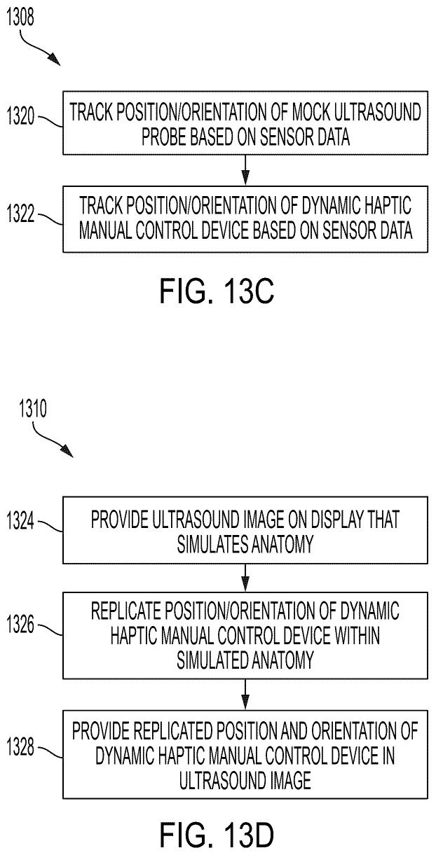

FIG. 13A depicts an illustrative method 1300 of providing an automated training system. With reference to FIGS. 1-12, the method 1300 is generally completed with the various components of the automated training system 100, particularly the computing devices 106 thereof. At block 1302, the method 1300 includes receiving one or more images from an imaging device (e.g., imaging devices 108) arranged such that a field of view of the imaging device includes a tray (e.g., tray 202) supporting one or more tools (e.g., tools 204) and a training surface (e.g., training surface 110) having a simulated subcutaneous area (e.g., simulated subcutaneous area 206). For example, the imaging device may transmit image data or the like via wired or wireless means to the computing device.

At block 1304, the method 1300 includes determining a location, a position, and an identification of the one or more tools supported on the tray. For example, as described herein, the determination is generally completed by utilization of image recognition software that is particularly configured to recognize items that typically would be located in the field of view of the imaging device and/or by utilizing a trained ML algorithm (e.g., such as one stored on machine learning devices 104) to recognize objects that may not otherwise be known or cannot be recognized due to variations, positioning, and/or the like. With reference to FIG. 13B, such a step may further include labeling tools supported on the tray based on the determined location, position, and identification at block 1316, as described herein. Such a step may also include, at block 1318, utilizing a ML computer vision algorithm to track movement of the tool and/or the dynamic haptic manual control device using the labels as the devices are moved by a user during a procedure, as described herein.

Referring again to FIG. 13A, at block 1306, the method 1300 includes receiving an input from a position tracking system (e.g., position tracking system 114). The input generally corresponds to various insertion characteristics of one of the tools and/or a dynamic haptic manual control device (e.g., the inner needle 422 within the simulated subcutaneous area 206, such as when the inner needle 422 pierces the pierceable area 206a and is inserted into the funnel system 206b and/or the false vein 206c).

At block 1308, the method 1300 further includes determining a positioning and an orientation of at least a portion of the tool and/or the dynamic haptic manual control device within the simulated subcutaneous area based on the insertion characteristics and the images of the training surface. For example, the computing devices 106 may combine the image data from the imaging devices 108 to determine the external location and orientation of the device that is inserted into the simulated subcutaneous area 206 and match the image data with data from the subcutaneous sensors 214 for determining the internal location and orientation of the device to develop an overall picture of the orientation and positioning of the device. Such a process may include interfacing with machine learning devices 104 that are trained to recognize the various data inputs, determine positioning and orientation, and develop an overall positioning and orientation estimation based on the combined data that is received. In some aspects, this process may be broken down into additional steps, as depicted in FIG. 13C. More specifically, at block 1320, the method 1300 may include tracking a position and an orientation of a mock ultrasound probe (e.g., the position tracking system 114) based on information received from a first electromagnetic position tracking sensor disposed on or near the mock ultrasound probe. Alternatively, the mock ultrasound probe may be an actual ultrasound probe that provides additional image data of the simulated subcutaneous area 206 that can be used for positioning and orientation determination, as described herein. At block 1322, the position and orientation of the dynamic haptic manual control device can be tracked in a similar fashion as noted above, by combining the various data streams (e.g., data from the imaging devices 108, data from the subcutaneous sensors 214, and data from the position tracking system 114) and utilizing a trained ML algorithm on the machine learning devices 104 to determine the position and orientation. The various data streams may be combined using a Kalman filter, a complimentary filter, and/or the like to make inferences regarding object tracking, location, positioning, engagement, and/or the like.

Returning to FIG. 13A, at block 1310, the method 1300 includes providing feedback, via a display (e.g., one of the interactive user interface devices 116, display 212, and/or one or more external computing devices, such as a proctor's computing device, a supervisor's computing device, and/or the like) and/or the dynamic haptic manual control device (e.g., via the LEDs 438 disposed on or in the hollow syringe body 402), regarding the positioning and orientation of the tool and/or the dynamic haptic manual control device. The feedback is not limited in this disclosure, and can generally be any feedback. For example, a user may not be able to advance to a next step in a process until certain feedback is received regarding various movements or actions. In another example, the feedback may be a grading of the user's overall strategy for a process or steps of a process. In still another example, the feedback may be in the form of illumination of the LEDs 438 indicating that simulated blood has been aspirated, as described herein. Various other feedback may also be given. For example, in addition to LED indicators, haptic vibration sensors on the device can indicate feedback to a user. In addition, feedback can be provided by mechanically loosening the haptic mechanism to release force on the aspirator (e.g., the syringe plunger 408). This haptic change provides the user feedback information that they have struck a simulated vein or artery. Other feedback is also contemplated, such as audible feedback, haptic feedback from haptic motors, and/or the like.

Referring now to FIG. 13D, block 1310 may further include various steps with respect to ultrasound imaging (e.g., by utilizing data from the position tracking system 114) to further provide feedback to a user. For example, at block 1324, an ultrasound image may be provided on the display 212, the image simulating a typical human anatomy that corresponds to the selected feedback profile. In addition, at block 1326, the position/orientation of the dynamic haptic manual control device based on the obtained data may be used to replicate an image of the same on the simulated ultrasound image, which can then be provided to the user at block 1328 (e.g., via the display 212).

Referring again to FIG. 13A, method 1300 may optionally include, at block 1312, determining one or more skill performance metrics of the user based on the obtained and processed data noted above, which is then optionally provided to the user (e.g., via the display 212) and/or one or more external devices, such as an external computing device, at block 1314. Such performance metrics may be certain metrics that have been developed working with medical professionals based on defined measures of success. For CVC, this may include, but is not limited to, an overall score, an angle of insertion, a position accuracy, a passing or not passing through the back of the vein, a striking or not striking the artery, a number of insertions, an amount of aspiration, and an amount of time visualizing the needle tip.

It should now be understood that the systems and methods described herein provide a training to users for completing procedures, particularly medical procedures such as CVC insertion procedures, by providing specialized tools that track user movements outside a simulated tissue area and inside a simulated subcutaneous area. This is completed using a combination of image data and data from various other sensors, such as electromagnetic based sensors, and utilizing machine learning to combine the data streams together to accurately determine positioning. Feedback is also provided to the user via a manual device that allows specific feedback profiles to be selected, as well as electronic feedback in the form of a display and/or LEDs on the manual device. As a result, users are able to obtain necessary training without fear of damage of tissue on living or deceased subjects, all while providing real-world simulation.

Further aspects of the present disclosure are provided by the subject matter of the following clauses:

Aspect 1: An automated training system, comprising: an imaging device arranged to capture one or more images of a tray supporting one or more tools and a training surface having a simulated subcutaneous area; a dynamic haptic manual control device; a position tracking system; a display; and a computing device communicatively coupled to the imaging device, the dynamic haptic manual control device, the position tracking system, and the display, the computing device configured to: receive the one or more images from the imaging device, determine a location, a position, and an identification of the one or more tools supported on the tray, receive an input from the position tracking system, the input corresponding to insertion characteristics of: a tool of the one or more tools into the training surface, and/or the dynamic haptic manual control device, determine, based on the insertion characteristics and the one or more images of the training surface, a positioning and an orientation of at least a portion of the tool and/or the dynamic haptic manual control device within the simulated subcutaneous area, and provide feedback, via the display and/or the dynamic haptic manual control device, regarding the positioning and orientation of the tool and/or the dynamic haptic manual control device.

Aspect 2: The automated training system according to aspect 1, wherein the dynamic haptic manual control device is a dynamic haptic syringe comprising a sensor and a retractable telescopic needle that is configured to provide force feedback for simulating needle insertion through the training surface into the simulated subcutaneous area.

Aspect 3: The automated training system according to aspect 2, wherein the force feedback is provided based on a selected profile on the dynamic haptic syringe.

Aspect 4: The automated training system according to aspect 2 or 3, wherein the dynamic haptic syringe comprises: a detector switch to track aspiration usage; and a light emitting diode (LED) that provides blood flash feedback.

Aspect 5: The automated training system according to any one of the preceding claims, wherein the training surface simulates one or more anatomical features of a subject.

Aspect 6: The automated training system according to any one of the preceding claims, wherein the simulated subcutaneous area comprises a funnel system coupled to a false vein comprising one or more subcutaneous sensors.

Aspect 7: The automated training system according to aspect 6, wherein the position tracking system comprises the one or more subcutaneous sensors.

Aspect 8: The automated training system according to any one of the preceding claims, wherein determining the positioning and the orientation of at least the portion of the tool and/or the dynamic haptic manual control device within the simulated subcutaneous area comprises: labeling each of the one or more tools supported on the tray based on the determined location, position, and identification of the one or more tools supported on the tray; and utilizing a machine learning computer vision algorithm to track movement of the tool and/or the dynamic haptic manual control device using the labels.

Aspect 9: The automated training system according to any one of the preceding claims, further comprising the one or more tools, wherein the one or more tools are blunted medical tools that are color tagged for computer vision detection.

Aspect 10: The automated training system according to any one of the preceding claims, wherein the feedback comprises instructions and warnings regarding usage and procedural order of the tool and/or the dynamic haptic manual control device.

Aspect 11: The automated training system according to any one of the preceding claims, wherein the position tracking system comprises a mock ultrasound probe having an electromagnetic position tracking sensor.

Aspect 12: The automated training system according to aspect 11, wherein the dynamic haptic manual control device comprises a second electromagnetic position tracking sensor.

Aspect 13: The automated training system according to aspect 11 or 12, wherein determining the positioning and the orientation further comprises: tracking a position and an orientation of the mock ultrasound probe based on information received from the electromagnetic position tracking sensor; and tracking a position and an orientation of the dynamic haptic manual control device based on information received from the second electromagnetic position tracking sensor.

Aspect 14: The automated training system according to aspect 13, wherein providing the feedback further comprises: providing an ultrasound image on the display, wherein the ultrasound image simulates an anatomy of a subject based on the positioning and orientation of the mock ultrasound probe; and replicate the position and orientation of the dynamic haptic manual control device within the simulated anatomy of the subject based on the positioning and orientation of the dynamic haptic manual control device; and provide the replicated position and orientation of the dynamic haptic manual control device in the ultrasound image on the display.

Aspect 15: The automated training system according to any of the preceding claims, wherein the computing device is further configured to: determine skill performance metrics based the positioning and orientation of the tool and/or the dynamic haptic manual control device; and provide the skill performance metrics via the display and/or an external device communicatively coupled to the computing device.

Aspect 16: The automated training system according to any one of the preceding claims, further comprising an interactive user interface that comprises the display, wherein the interactive user interface provides one or more user interface controls via the display to a user.

Aspect 17: An automated training system, comprising: a computing device; and a non-transitory, computer-readable storage medium communicatively coupled to the computing device, the non-transitory, computer-readable storage medium comprising one or more programming instructions thereon that, when executed, cause the computing device to: receive one or more images from an imaging device arranged such that a field of view of the imaging device includes a tray supporting one or more tools and a training surface having a simulated subcutaneous area, determine a location, a position, and an identification of the one or more tools supported on the tray, receive an input from a position tracking system, the input corresponding to insertion characteristics of: a tool of the one or more tools into the training surface, and/or a dynamic haptic manual control device, determine, based on the insertion characteristics and the one or more images of the training surface, a positioning and an orientation of at least a portion of the tool and/or the dynamic haptic manual control device within the simulated subcutaneous area, and provide feedback, via a display and/or the dynamic haptic manual control device, regarding the positioning and orientation of the tool and/or the dynamic haptic manual control device.

Aspect 18: The automated training system according to aspect 17, wherein providing the feedback via the dynamic haptic manual control device comprises causing the dynamic haptic manual control device to emit light via a light emitting diode (LED) disposed on the dynamic haptic manual control device.

Aspect 19: The automated training system according to aspect 17 or 18, wherein providing the feedback via the dynamic haptic manual control device comprises causing the dynamic haptic manual control device to provide force feedback to a user holding the dynamic haptic manual control device.

Aspect 20: The automated training system according to any one of aspects 17 to 19, wherein determining the positioning and the orientation of at least the portion of the tool and/or the dynamic haptic manual control device within the simulated subcutaneous area comprises: labeling each of the one or more tools supported on the tray based on the determined location, position, and identification of the one or more tools supported on the tray; and utilizing a machine learning computer vision algorithm to track movement of the tool and/or the dynamic haptic manual control device using the labels.

Aspect 21: The automated training system according to any one of aspects 17 to 20, wherein the feedback comprises instructions and warnings regarding usage and procedural order of the tool and/or the dynamic haptic manual control device.

Aspect 22: The automated training system according to any one of aspects 17 to 21, wherein determining the positioning and the orientation further comprises: tracking a position and an orientation of the mock ultrasound probe based on information received from a first electromagnetic position tracking sensor disposed on the mock ultrasound probe; and tracking a position and an orientation of the dynamic haptic manual control device based on information received from a second electromagnetic position tracking sensor disposed on the dynamic haptic manual control device.

Aspect 23: The automated training system according to aspect 22, wherein providing the feedback further comprises: providing an ultrasound image on the display, wherein the ultrasound image simulates an anatomy of a subject based on the positioning and orientation of the mock ultrasound probe; and replicate the position and orientation of the dynamic haptic manual control device within the simulated anatomy of the subject based on the positioning and orientation of the dynamic haptic manual control device; and provide the replicated position and orientation of the dynamic haptic manual control device in the ultrasound image on the display.

Aspect 24: The automated training system according to any one of aspects 17 to 23, wherein the computing device is further configured to: determine skill performance metrics based the positioning and orientation of the tool and/or the dynamic haptic manual control device; and provide the skill performance metrics via the display and/or an external device communicatively coupled to the computing device.

Aspect 25: A method of providing an automated training system, comprising: receiving, by a computing device, one or more images from an imaging device arranged such that a field of view of the imaging device includes a tray supporting one or more tools and a training surface having a simulated subcutaneous area, determining, by the computing device, a location, a position, and an identification of the one or more tools supported on the tray, receiving, by a computing device, an input from a position tracking system, the input corresponding to insertion characteristics of: a tool of the one or more tools into the training surface, and/or a dynamic haptic manual control device, determining, based on the insertion characteristics and the one or more images of the training surface, a positioning and an orientation of at least a portion of the tool and/or the dynamic haptic manual control device within the simulated subcutaneous area, and providing feedback, via a display and/or the dynamic haptic manual control device, regarding the positioning and orientation of the tool and/or the dynamic haptic manual control device.

Aspect 26: The method according to aspect 25, wherein providing the feedback via the dynamic haptic manual control device comprises causing the dynamic haptic manual control device to emit light via a light emitting diode (LED) disposed on the dynamic haptic manual control device.

Aspect 27: The method according to aspect 25 or 26, wherein providing the feedback via the dynamic haptic manual control device comprises causing the dynamic haptic manual control device to provide force feedback to a user holding the dynamic haptic manual control device.

Aspect 28: The method according to any one of aspects 25 to 27, wherein determining the positioning and the orientation of at least the portion of the tool and/or the dynamic haptic manual control device within the simulated subcutaneous area comprises: labeling each of the one or more tools supported on the tray based on the determined location, position, and identification of the one or more tools supported on the tray; and utilizing a machine learning computer vision algorithm to track movement of the tool and/or the dynamic haptic manual control device using the labels.

Aspect 29: The method according to any one of aspects 25 to 28, wherein the feedback comprises instructions and warnings regarding usage and procedural order of the tool and/or the dynamic haptic manual control device.

Aspect 30: The method according to any one of aspects 25 to 29, wherein determining the positioning and the orientation further comprises: tracking a position and an orientation of the mock ultrasound probe based on information received from a first electromagnetic position tracking sensor disposed on the mock ultrasound probe; and tracking a position and an orientation of the dynamic haptic manual control device based on information received from a second electromagnetic position tracking sensor disposed on the dynamic haptic manual control device.

Aspect 31: The method according to aspect 30, wherein providing the feedback further comprises: providing an ultrasound image on the display, wherein the ultrasound image simulates an anatomy of a subject based on the positioning and orientation of the mock ultrasound probe; and replicate the position and orientation of the dynamic haptic manual control device within the simulated anatomy of the subject based on the positioning and orientation of the dynamic haptic manual control device; and provide the replicated position and orientation of the dynamic haptic manual control device in the ultrasound image on the display.

Aspect 32: The method according to any one of aspects 25 to 31, wherein the computing device is further configured to: determine skill performance metrics based the positioning and orientation of the tool and/or the dynamic haptic manual control device; and provide the skill performance metrics via the display and/or an external device communicatively coupled to the computing device.

Aspect 33: A dynamic haptic syringe apparatus for providing automated training, the dynamic haptic syringe apparatus comprising: a hollow syringe body having an open proximal end and a distal end; a syringe plunger having a plunger body defining a distal end and a proximal end, the syringe plunger received within the open proximal end of the hollow syringe body and movable within the hollow syringe body; a hub removably coupled to the distal end of the hollow syringe body; a telescopic needle assembly coupled to the hub, the telescopic needle assembly comprising a hollow outer needle having an open distal end and an open proximal end having an end effector disposed thereon, and an inner needle comprising an elongate body having a distal end and a proximal end rigidly coupled to the hub, the elongate body received within the hollow outer needle, the inner needle fixed in position and the hollow outer needle movable between an extended position and a retracted position, wherein in the extended position, the proximal end of the hollow outer needle is disposed within the hub or adjacent to the hub and in the retracted position, the proximal end of the hollow outer needle extends through the hub into the hollow syringe body; and one or more slip flexures disposed within the hollow syringe body, the one or more slip flexures engagable with the end effector on the proximal end of the hollow outer needle to provide simulated feedback.

Aspect 34: The dynamic haptic syringe according to aspect 33, wherein the one or more slip flexures comprises a plurality of slip flexures, each one of the plurality of slip flexures shaped to provide different feedback profiles when engaged with the end effector on the proximal end of the elongate body of the inner needle.

Aspect 35: The dynamic haptic syringe according to aspect 34, wherein: each one of the plurality of slip flexures are independently movable between an engaged position and a disengaged position, in the engaged position, the slip flexure engages with the end effector to provide the feedback profile, and in the disengaged position, the slip flexure does not engage with the end effector and does not provide the feedback profile.

Aspect 36: The dynamic haptic syringe according to aspect 35, wherein each one of the plurality of slip flexures is movable between the engaged position and the disengaged position via a selection ring.

Aspect 37: The dynamic haptic syringe according to any one of aspects 33 to 36, further comprising at least one sensor configured to sense an insertion of the distal end of the elongate body of the inner needle within a training surface and provide sensor data corresponding to the insertion.

Aspect 38: The dynamic haptic syringe according to aspect 37, wherein the at least one sensor is further configured to provide the sensor data to a computing device.

Aspect 39: The dynamic haptic syringe according to aspect 37, wherein the at least one sensor is a Hall effect sensor.

Aspect 40: The dynamic haptic syringe according to any one of aspects 33 to 39, further comprising a light emitting diode (LED) disposed on or in the syringe body or the hub.

Aspect 41: The dynamic haptic syringe according to aspect 40, wherein the LED is configured to illuminate based on signals received from a computing device.

Aspect 42: The dynamic haptic syringe according to any one of aspects 33 to 41, wherein the plunger body of the syringe plunger defines a lumen extending between the distal end and the proximal end thereof, the lumen configured to receive a guidewire therein.

Aspect 43: The dynamic haptic syringe according to any one of aspects 33 to 41, wherein the hub is configured to be disconnected from the hollow syringe body after insertion of the needle assembly into a training surface and couplable to a guidewire delivery mechanism, the guidewire delivery mechanism comprising a guidewire that is received within the telescopic needle assembly and is advanced distally through the telescopic needle assembly into the training surface.

Aspect 44: An automated training system, comprising: a training surface having a simulated subcutaneous area; and the dynamic haptic syringe according to any one of aspects 33 to 43, wherein the distal end of the inner needle is insertable into the simulated subcutaneous area.

Aspect 45: The automated training system according to aspect 44, further comprising an imaging device arranged to capture one or more images of the training surface.

Aspect 46: The automated training system according to aspect 44 or 45, further comprising a position tracking system.

Aspect 47: The automated training system according to any one of aspects 44 to 46, further comprising a display and a computing device communicatively coupled to the display, the computing device configured to monitor insertion of the distal end of the inner needle into the simulated subcutaneous area and provide feedback via the display and/or the dynamic haptic syringe.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. An automated training system, comprising:
a computing device; and
a non-transitory, computer-readable storage medium communicatively coupled to the computing device, the non-transitory, computer-readable storage medium comprising one or more programming instructions thereon that, when executed, cause the computing device to:
receive one or more images from an imaging device arranged such that a field of view of the imaging device includes a tray supporting one or more tools and a training surface having a simulated subcutaneous area,
determine a location, a position, and an identification of the one or more tools supported on the tray,
receive an input from a position tracking system, the input corresponding to insertion characteristics of a tool of the one or more tools into the training surface,
determine, based on the insertion characteristics and the one or more images of the training surface, a positioning and an orientation of at least a portion of the tool within the simulated subcutaneous area, and
provide feedback regarding the positioning and orientation of the tool.

2. The automated training system according to claim 1, wherein determining the position and orientation of at least the portion of the tool comprises determining based on feedback from a dynamic haptic manual control device.

3. The automated training system according to claim 2, wherein providing the feedback comprises causing the dynamic haptic manual control device to emit light via a light emitting diode (LED) disposed on the dynamic haptic manual control device.

4. The automated training system according to claim 2, wherein providing the feedback comprises emitting a sound, emitting one or more haptic vibrations, or a combination thereof.

5. The automated training system according to claim 1, wherein determining the positioning and the orientation of at least the portion of the tool within the simulated subcutaneous area comprises:
labeling each of the one or more tools supported on the tray based on the determined location, position, and identification of the one or more tools supported on the tray; and
utilizing a machine learning computer vision algorithm to track movement of the tool using the labels.

6. The automated training system according to claim 1, wherein the feedback comprises instructions and warnings regarding usage and procedural order of the tool.

7. The automated training system according to claim 1, wherein determining the positioning and the orientation further comprises:
tracking a position and an orientation of a mock ultrasound probe based on information received from a first electromagnetic position tracking sensor disposed on the mock ultrasound probe; and
tracking a position and an orientation of a dynamic haptic manual control device based on information received from a second electromagnetic position tracking sensor disposed on the dynamic haptic manual control device.

8. The automated training system according to claim 7, wherein providing the feedback further comprises:
providing an ultrasound image on a display, wherein the ultrasound image simulates an anatomy of a subject based on the positioning and orientation of the mock ultrasound probe; and
replicate the position and orientation of the dynamic haptic manual control device within the simulated anatomy of the subject based on the positioning and orientation of the dynamic haptic manual control device; and
provide the replicated position and orientation of the dynamic haptic manual control device in the ultrasound image on the display.

9. The automated training system according to claim 1, wherein the computing device is further configured to:
determine skill performance metrics based the positioning and orientation of the tool; and
provide the skill performance metrics via the display and/or an external device communicatively coupled to the computing device.

10. A method of providing an automated training system, comprising:
receiving, by a computing device, one or more images from an imaging device arranged such that a field of view of the imaging device includes a tray supporting one or more tools and a training surface having a simulated subcutaneous area;
determining, by the computing device, a location, a position, and an identification of the one or more tools supported on the tray;
receiving, by a computing device, an input from a position tracking system, the input corresponding to insertion characteristics of a tool of the one or more tools into the training surface;
determining, based on the insertion characteristics and the one or more images of the training surface, a positioning and an orientation of at least a portion of the tool within the simulated subcutaneous area; and
providing feedback regarding the positioning and orientation of the tool.

11. The method according to claim 10, wherein the input further corresponds to insertion characteristics of a dynamic haptic manual control device.

12. The method according to claim 11, wherein providing the feedback comprises causing the dynamic haptic manual control device to emit light via a light emitting diode (LED) disposed on the dynamic haptic manual control device.

13. The method according to claim 11, wherein providing the feedback comprises emitting a sound, emitting one or more haptic vibrations, or a combination thereof.

14. The method according to claim 10, wherein determining the positioning and the orientation of at least the portion of the tool within the simulated subcutaneous area comprises:
labeling each of the one or more tools supported on the tray based on the determined location, position, and identification of the one or more tools supported on the tray; and
utilizing a machine learning computer vision algorithm to track movement of the tool using the labels.

15. The method according to claim 10, wherein the feedback comprises instructions and warnings regarding usage and procedural order of the tool.

16. The method according to claim 10, wherein determining the positioning and the orientation further comprises:
tracking a position and an orientation of a mock ultrasound probe based on information received from a first electromagnetic position tracking sensor disposed on the mock ultrasound probe; and
tracking a position and an orientation of a dynamic haptic manual control device based on information received from a second electromagnetic position tracking sensor disposed on the dynamic haptic manual control device.

17. The method according to claim 16, wherein providing the feedback further comprises:
providing an ultrasound image on a display, wherein the ultrasound image simulates an anatomy of a subject based on the positioning and orientation of the mock ultrasound probe; and
replicate the position and orientation of the dynamic haptic manual control device within the simulated anatomy of the subject based on the positioning and orientation of the dynamic haptic manual control device; and
provide the replicated position and orientation of the dynamic haptic manual control device in the ultrasound image on the display.

18. The method according to claim 10, further comprising:
determining skill performance metrics based the positioning and orientation of the tool; and
providing the skill performance metrics via the display and/or an external device communicatively coupled to the computing device.

19. An automated training system, comprising:
an imaging device arranged to capture one or more images of a tray supporting one or more tools and a training surface having a simulated subcutaneous area;
a dynamic haptic manual control device;
a position tracking system;
a display; and
a computing device communicatively coupled to the imaging device, the dynamic haptic manual control device, the position tracking system, and the display, the computing device configured to:
receive the one or more images from the imaging device, determine a location, a position, and an identification of the one or more tools supported on the tray,
receive an input from the position tracking system, the input corresponding to insertion characteristics of: a tool of the one or more tools into the training surface, and/or the dynamic haptic manual control device,
determine, based on the insertion characteristics and the one or more images of the training surface, a positioning and an orientation of at least a portion of the tool and/or the dynamic haptic manual control device within the simulated subcutaneous area, and
provide feedback, via the display and/or the dynamic haptic manual control device, regarding the positioning and orientation of the tool and/or the dynamic haptic manual control device.

20. The automated training system of claim 19, wherein the training surface simulates one or more anatomical features of a subject.
